(19) United States
(12) Patent Application Publication
(10) Pub. No.: US 2026/0182880 A1
(43) Pub. Date: Jul. 2, 2026

(54) APPARATUS AND METHOD FOR EEG-BASED EMOTION CLASSIFICATION USING INTRINSIC MODE FUNCTIONS (IMFs) AND CONTRASTIVE PREDICTIVE CODING (CPC)

(71) Applicant: EMMA HEALTHCARE CO., LTD., Seongnam-si (KR)

(72) Inventor: Ryanghee SOHN, Seongnam-si (KR)

(73) Assignee: EMMA HEALTHCARE CO., LTD., Seongnam-si (KR)

(21) Appl. No.: 19/418,611

(22) Filed: Dec. 12, 2025

(30) Foreign Application Priority Data
Dec. 27, 2024 (KR) 10-2024-0198723

(51) Int. Cl. A61B 5/16; A61B 5/00; A61B 5/372; G16H 50/20

(52) U.S. Cl. CPC A61B 5/165; A61B 5/0006; A61B 5/372; A61B 5/7267; G16H 50/20

(57) ABSTRACT

The apparatus for emotion classification according to the present application comprises a sensor configured to acquire one or more electroencephalogram (EEG) signals of a user; and a processor operably coupled to the sensor, and configured to perform a bidimensional empirical mode decomposition (BEMD) process on the acquired one or more EEG signals to extract one or more intrinsic mode functions (IMFs), convert signal data of each of the extracted IMFs into one or more images, and classify an emotion of the user by applying the converted one or more images to a certain trained contrastive predictive coding (CPC)-based artificial intelligence model.

### BACKGROUND OF THE INVENTION

### Field of the Invention

This application claims the benefit of Korean Patent Application No. 10-2024-0198723, filed on Dec. 27, 2024, which is hereby incorporated by reference as if fully set forth herein. The present invention relates to apparatus and method for EEG-based emotion classification using intrinsic mode functions (IMFs) and contrastive predictive coding (CPC). This invention was made as part of the research project titled ‘Development of a Quantitative emotion-affective evaluation model for users in non-face-to-face environments and commercialization for bidirectional digital content applications,’ supported by the Ministry of Science and ICT and the Institute for Information & Communications Technology Planning & Evaluation (IITP) under the Realistic Content Core Technology Development Program (Project No. 2710008164), and was conducted during the period from Jan. 1, 2025 to Dec. 31, 2025.

### DESCRIPTION OF THE RELATED ART

Conventional emotion classification apparatuses have extracted simple features such as band-specific power or statistical characteristics from EEG signals. However, such features have limitations in that they fail to sufficiently capture the nonlinear and nonstationary characteristics of EEG signals. In particular, considering that brain activity patterns vary dynamically over time depending on emotional states and that multiple brain regions interact in a highly complex manner, simple feature-extraction approaches are inadequate for effectively capturing such complex patterns.

Furthermore, existing supervised learning-based artificial intelligence techniques require a large amount of labeled data. In the case of emotion-related data, however, it is extremely difficult to obtain reliable training data because accurate labeling is challenging and subjective factors are significantly involved. In addition, emotional expressions differ from person to person, and even the same emotion may exhibit different patterns depending on the situation. As a result, conventional supervised learning approaches have difficulty effectively handling such individual differences and context-dependent variations.

### SUMMARY OF THE INVENTION

It is an object of the present invention to provide an emotion classification apparatus and emotion classification method based on EEG technology.

In accordance with an aspect of the present invention, the above and other objects can be accomplished by the provision of an emotion classification apparatus according to the present invention may comprise a sensor configured to acquire one or more electroencephalogram (EEG) signals of a user; and a processor operably coupled to the sensor, and configured to: perform a bidimensional empirical mode decomposition (BEMD) process on the acquired one or more EEG signals to extract one or more intrinsic mode functions (IMFs), convert signal data of each of the extracted IMFs into one or more images, and classify an emotion of the user by applying the converted one or more images to a certain trained contrastive predictive coding (CPC)-based artificial intelligence model.

The sensor is further configured to acquire the EEG signals from at least one region corresponding to Fp1, Fp2, AF3, AF4, F3, F4, F7, and F8 of an International 10-20 electrode placement system.

The processor is further configured to perform the BEMD process by pairing EEG signals acquired from positions symmetrical with respect to a midline of a skull among the one or more EEG signals acquired through the sensor.

The processor is further configured to convert the extracted IMFs into an image including at least one of a sampling interval, time, amplitude, and frequency.

The processor is further configured to perform at least one preprocessing operation selected from a noise removal, a frequency filtering, and a signal normalization on the one or more EEG signals acquired through the sensor prior to performing the BEMD process.

In accordance with an aspect of the present invention, the above and other objects can be accomplished by the provision of an emotion classification apparatus according to the present invention may comprise a communication module configured to receive one or more electroencephalogram (EEG) signals of a user; and a processor operably coupled to the communication module, and configured to: perform a bidimensional empirical mode decomposition (BEMD) process on the received one or more EEG signals to extract one or more intrinsic mode functions (IMFs); convert signal data of each of the extracted IMFs into one or more images; and classify an emotion of the user by applying the converted one or more images to a certain trained contrastive predictive coding (CPC)-based artificial intelligence model.

In accordance with an aspect of the present invention, the above and other objects can be accomplished by the provision of method for emotion classification by an emotion classification apparatus comprises acquiring one or more electroencephalogram (EEG) signals of a user; performing a bidimensional empirical mode decomposition (BEMD) process on the acquired one or more EEG signals to extract one or more intrinsic mode functions s (IMFs); converting signal data of each of the extracted IMFs into one or more images; and classifying an emotion of the user by applying the converted one or more images to a certain trained contrastive predictive coding (CPC)-based artificial intelligence model.

The acquiring comprises acquiring the EEG signals from at least one region corresponding to Fp1, Fp2, AF3, AF4, F3, F4, F7, and F8 of an International 10-20 electrode placement system.

The performing comprises performing the BEMD process by pairing EEG signals acquired from positions symmetrical with respect to a midline of a skull among the one or more EEG signals.

The converting comprises converting the extracted IMFs into an image including at least one of a sampling interval, time, amplitude, and frequency.

The method further comprises performing at least one preprocessing operation selected from a noise removal, a frequency filtering, and a signal normalization on the one or more EEG signals acquired through the sensor prior to performing the BEMD process.

In accordance with an aspect of the present invention, the above and other objects can be accomplished by the provision of method for emotion classification by an emotion classification apparatus comprises receiving one or more electroencephalogram (EEG) signals of a user; performing a bidimensional empirical mode decomposition (BEMD) process on the received one or more EEG signals to extract one or more intrinsic mode functions (IMFs); converting signal data of each of the extracted IMFs into one or more images; and classifying an emotion of the user by applying the converted one or more images to a certain trained contrastive predictive coding (CPC)-based artificial intelligence model.

In accordance with an aspect of the present invention, the above and other objects can be accomplished by the provision of non-transitory computer-readable storage medium storing instructions that, when executed by one or more processors, cause the one or more processors to: acquire one or more electroencephalogram (EEG) signals of a user; perform a bidimensional empirical mode decomposition (BEMD) process on the acquired one or more EEG signals to extract one or more intrinsic mode functions (IMFs); convert signal data of each of the extracted IMFs into one or more images; and classify an emotion of the user by applying the converted one or more images to a certain trained contrastive predictive coding (CPC)-based artificial intelligence model.

In accordance with an aspect of the present invention, the above and other objects can be accomplished by the provision of non-transitory computer-readable storage medium storing instructions that, when executed by one or more processors, cause the one or more processors to: receive one or more electroencephalogram (EEG) signals of a user; perform a bidimensional empirical mode decomposition (BEMD) process on the received one or more EEG signals to extract one or more intrinsic mode functions (IMFs); convert signal data of each of the extracted IMFs into one or more images; and classify an emotion of the user by applying the converted one or more images to a certain trained contrastive predictive coding (CPC)-based artificial intelligence model.

### BRIEF DESCRIPTION OF THE DRAWINGS

The accompanying drawings, which are incorporated in and constitute a part of the detailed description, provide exemplary embodiments of the present invention and, together with the detailed description, serve to explain the technical spirit of the invention.

FIG. 1 is a view illustrating a layered structure of an artificial neural network.

FIG. 2 is a view showing an example of a deep neural network.

FIG. 3 is a diagram illustrating an example of a data processing procedure of a contrastive predictive coding (CPC)-based artificial intelligence model.

FIG. 4 is a block diagram illustrating the functional configuration of an EEG-based emotion classification apparatus 400 according to an embodiment of the present invention.

FIG. 5 is a diagram illustrating an example of a data processing procedure of the EEG-based emotion classification apparatus 400 according to an embodiment of the present invention.

FIG. 6 is a diagram illustrating the structure of an International 10-20 electrode placement system.

FIG. 7 is a diagram illustrating an example of a bidimensional empirical mode decomposition (BEMD) process of the EEG-based emotion classification apparatus 400 according to the present invention.

FIG. 8 is another exemplary diagram illustrating a block diagram of an EEG-based emotion classification apparatus 800 according to the present invention.

FIG. 9 is a diagram illustrating performance evaluation results of the EEG-based emotion classification apparatuses 400 and 800 according to the present invention.

### DETAILED DESCRIPTION OF THE INVENTION

The present invention may be embodied in various forms and may have multiple embodiments. Specific embodiments are illustrated in the drawings and will be described in detail; however, these are presented merely to exemplify the invention, and are not intended to limit the invention to particular embodiments. It should be understood that all modifications, equivalents, and alternatives falling within the spirit and scope of the invention are intended to be encompassed.

When an element is described as being connected to′ or ‘coupled to’ another element, the element may be directly connected or coupled to the other element, or other intervening elements may be present. In contrast, when an element is described as being ‘directly connected to’ or ‘directly coupled to’ another element, no intervening elements are present.

The terms ‘first,’ ‘second,’ and the like may be used to describe various elements, but such terms shall not limit the elements by their numerical designation. These terms are used merely to distinguish one element from another.

The terminology used herein is for the purpose of describing particular embodiments only and is not intended to limit the invention. As used herein, the singular forms include the plural forms unless the context clearly indicates otherwise. The terms ‘include’ and ‘have,’ and any variations thereof, are intended to specify the presence of stated features, integers, steps, operations, components, or combinations thereof, but do not preclude the presence or addition of one or more other features, integers, steps, operations, components, or combinations thereof.

Further, the terms ‘unit,’ ‘module,’ ‘part,’ or ‘device,’ as used herein, refer to a functional element configured to perform at least one operation or function, and may be implemented in hardware, software, or a combination thereof.

In some cases, well-known structures and devices may be omitted or shown in block diagram form to avoid obscuring the concept of the invention. Throughout the drawings, identical reference numerals denote identical or similar components.

The components described in connection with the embodiments with reference to the drawings are not necessarily limited to the specific embodiments, and other embodiments may include the components so long as the technical spirit of the invention is maintained. Multiple embodiments may also be combined into a single embodiment, even if not expressly described.

Before describing the present invention, artificial intelligence (AI), machine learning, and deep learning will be described. As a method of most easily understanding the relationship among the three concepts, three concentric circles may be imagined. Artificial intelligence may be the outermost circle, machine learning may be the middle circle, and deep learning, which leads a current artificial intelligence boom, may be the innermost circle.

The concept of artificial intelligence first appeared in the Dartmouth workshop held by Professor John Mccarthy at Dartmouth College, USA in the year of 1956, and has explosively grown in recent years. In particular, artificial intelligence has been further accelerated as the result of introduction of a GPU, which has provided rapid and strong parallel processing performance since 2015. The advent of the big data era with ever-expanding storage capacity and numerous data in all areas, such as images, text, and mapping data, had a great influence on such growth of artificial intelligence.

### Artificial Intelligence-Human Intelligence Implemented by Machine

In 1956, artificial intelligence pioneers dreamt of manufacturing a complex computer having similar characteristics to human intelligence. Artificial intelligence that thinks like a human being while having sense and thinking power of the human being is called a “general AI”, whereas artificial intelligence that can be made at the level of the current technological advancement is included in the concept of “narrow AI”. Narrow AI is characterized in that it is possible to perform specific tasks, such as an image sorting service or a facial recognition function on social media, with greater than human ability.

### Machine Learning-Specific Approach That Implements Artificial Intelligence

Machine learning serves to automatically filter spam in a mailbox. Meanwhile, basically, machine learning analyzes data using an algorithm, learns through analysis, and performs determination or prediction based on what has been learned. Ultimately, therefore, machine learning aims to “train” a computer itself using a large amount of data and the algorithm so as to learn a task execution method, instead of specific guidelines for decision making being directly coded in software. Machine learning came from the concept that early artificial intelligence researchers directly advocated, and decision tree learning, inductive logic programming, clustering, reinforcement learning, and a Bayesian network are included in algorithm schemes. However, none thereof has achieved general AI, which is the final target, and there were many cases in which it was difficult to complete even narrow AI using an initial machine learning approach.

Although machine learning is making great achievements in the field of computer vision at the present time, machine learning encountered the limitation in that a predetermined amount of coding work is accompanied over a process of implementing artificial intelligence, even if there are no specific guidelines. When an image of a stop sign is recognized using a machine learning system, for example, a developer must directly manufacture a border sensing filter that identifies a start part and an end part of an object using a program, shape sensing that determines the surface of the object, and a classifier that recognizes letters such as “S-T-O-P” by coding. Like this, machine learning is operated in a manner in which the image is recognized from the “coded” classifier and the stop sign is “learned” through an algorithm.

A machine learning training method finds a most appropriate model by adjusting parameters of a model so that an error between a target value and a predictive value is minimized. Here, the predictive value refers to a value output when an input value is entered into a model, that is, an output value. For example, a model including an arbitrary number of convolution layers, bidirectional LSTMS, feedforward layers, etc. changes each of the convolution layers, bidirectional LSTMs, and feedforward layers so that an error with respect to a target value is minimized as training progresses.

An image recognition rate of machine learning is sufficient in performance to be commercialized. In a specific situation in which the sign is invisible due to fog or trees, however, the image recognition rate of machine learning may be reduced. The reason that computer vision and image recognition have not reached to the level of the human being until recently is because of such a recognition rate problem and frequent errors.

### Deep Learning-Technology That Implements Complete Machine Learning

What gave inspiration to an artificial neural network, which is another algorithm that early machine learning researchers made, is the biological characteristics of a human brain, particularly a neuron connection structure. However, the artificial neural network has uniform layer connection and data propagation direction, unlike the brain in which physically adjacent neurons can be connected to each other.

For example, when an image is cut into a great number of tiles and the tiles are input to a first layer of the neural network, the neurons repeat a process of transmitting data to the next layer until a final output is generated by the last layer. A weight indicating input accuracy based on a task that is performed is assigned to each neuron, and after that, all weights are summed, whereby final output is determined. For the stop sign, characteristics of the image, such as the octagonal shape, red color, displayed letters, size, and motion thereof, are finely cut and “inspected”, by the neurons, and the duty of the neural network is to determine whether this is a stop sign. Here, a “provability vector” that predicts the result according to the weights based on sufficient data is utilized.

Deep learning, which is artificial intelligence developed from an artificial neural network, learns data utilizing information input and output layers similar to neurons of a brain. Since even a basic neural network required an awesome amount of operation, however, commercialization of deep learning faced difficulties from the beginning. Nevertheless, research has continued, and parallelization of an algorithm improving the concept of deep learning based on a super computer was successful. The advent of a GPU optimized for parallel operation has epochally accelerated the operating speed of the neural network, whereby artificial intelligence based on true deep learning appeared.

There is a high possibility of the neural network giving a great number of wrong answers during “learning”. Back to the example of the stop sign, hundreds, thousands, or millions of images may be learned in order to accurately adjust neuron input weights so as to always give correct answers irrespective of weather conditions and change of day and night. It can be seen that the neural network has sufficiently learned the stop sign only when this level of accuracy was reached. In the year of 2012, Google and Professor Andrew NG at Stanford University implemented a “deep neural network” constituted by about one billion or more of neurons using 16,000 computers. 10 million images were picked and analyzed from YouTube therethrough, and the computers succeeded in classifying images of people and images of cats. The computers learned a process of recognizing and determining the shape and appearance of the cats displayed on the screens by themselves.

Image recognition ability of a system trained through deep learning has already gone ahead of a human being. In addition, ability of recognizing cancer cells in blood and ability of recognizing tumors through MRI scanning are included in the deep learning area. AlphaGo of Google learned the fundamentals of baduk, which is a Korean strategy board game, and further strengthened the neural network while repeatedly playing games with AI like itself. As the result of the advent of deep learning, practicality of machine learning has been reinforced, and the artificial intelligence area was extended. Deep learning subdivides a task in all supportable manners through a computer system. Deep learning-based technologies, such as a car without a driver, better preventive healthcare, and more accurate movie recommendation, have already been used in our daily life or are about to be put into practice. Deep learning is evaluated as the present and future of artificial intelligence having potential power capable of realizing general AI that appeared in science fiction.

Hereinafter, deep learning will be described in more detail.

Deep learning, which is a kind of artificial neural network (ANN) using a human neural network theory, is a machine learning model or an algorithm set referring to a deep neural network (DNN) configured to have a layered structure in which at least one hidden layer (hereinafter referred to as an “intermediate layer”) is provided between an input layer and an output layer. Briefly, deep learning may be an artificial neural network having deep layers.

A human brain is estimated to be constituted by 25 billion nerve cells, each nerve cell (neuron) refers to one neuron constituting the neural network. One neuron includes one cell body, one axon or nurite, which is a protrusion of the cell body, and several dendrites or protoplasmic processes. Information exchange between neurons is performed through synapse, which is a junction between neurons. Although one neuron is very simple, a group of neurons may have human intelligence. The dendrites are inputs configured to receive signals sent by other neurons, and the axon, which is a portion extending from the cell body, is an output configured to transmit a signal to another neuron. The synapse is a connection portion configured to connect the axon and the dendrites, which transmit signals between neurons, to each other. Signals of the neurons are not unconditionally transmitted but are transmitted only when the intensity of each signal is a predetermined value (threshold) or more. That is, synapses have different connection intensities, and each synapse determines whether to transmit a signal.

An artificial neural network (ANN), which is one field of artificial intelligence, is a mathematical model modeled by imitating the brain structure (neural network) of biology (generally a human being). That is, the artificial neural network is implemented by imitating an information processing and transmission process of a biological neuron. The artificial neural network is implemented similarly to a manner in which a human brain solves problems, and the neural network has excellent parallelism, since the neurons independently operate. In addition, since information is dispersed in many connection lines, no great influence is exerted on all neurons even though some of the neurons have problems, and therefore the artificial neural network is resistant to a predetermined level of errors and has learning ability in a given environment.

A deep neural network, which is a descendant of the artificial neural network, is the latest version of the artificial neural network that goes beyond the existing limits and has achieved successes in areas in which a large number of artificial intelligence technologies suffered failures in the past. When describing modeling an artificial neural network by imitating a biological neural network, biological neurons are modeled as nodes in terms of processing units, and synapses are modeled as weights in terms of connections, as shown in Table 1 below.

TABLE 1

| Biological neural network | Artificial neural network |
|---|---|
| Cell body | Node |
| Dendrite | Input |
| Axon | Output |
| Synapse | Weight |

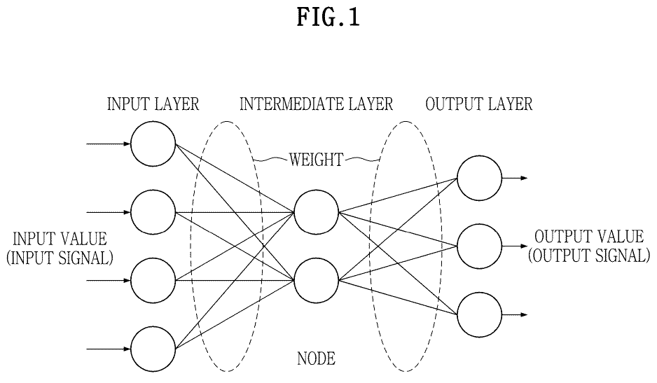

FIG. 1 is a view illustrating a layered structure of an artificial neural network.

Like a plurality of biological neurons of a human being, not a single biological neuron, is connected to each other in order to perform a meaningful task, for an artificial neural network, individual neurons are also connected to each other via synapses, whereby a plurality of layers is connected to each other, wherein connection intensity between the respective layers may be updated using weights. The multilayered structure and connection intensity are utilized in a field for learning and recognition.

The respective nodes are connected to each other via links having weights, and the entire model performs learning while repeatedly adjusting weights. The weights, which are basic means for long-term memory, express importance of the respective nodes. The artificial neural network initializes the weights and updates and adjusts weights using a data set to be trained in order to train the entire model. When a new input value is input after training is completed, an appropriate output value is inferred. The learning principle of the artificial neural network is a process in which intelligence is formed through generalization of experiences, and learning is performed in a bottom-up manner. When two or more (i.e. 5 to 10) intermediate layers are provided, as shown in FIG. 1, this means that the layers are deepened and is called a deep neural network, and a learning and inference model achieved through the deep neural network may be referred to as deep learning.

The artificial neural network may play a role to some extent even when the artificial neural network has one intermediate layer (generally referred to as a “hidden layer”) excluding input and output. When problem complexity increases, however, the number of nodes or the number of layers must be increased. It is effective to increase the number of layers so as to provide a multilayered model; however, an available range is restrictive due to limitations in that efficient learning is impossible and the amount of calculation necessary to train the network is large.

As a result of overcoming existing limitations described above, however, the artificial neural network was configured to have a deep structure. Consequently, a complex and expressive model has been constructed, and epochal results have been announced in various fields, such as voice recognition, facial recognition, object recognition, and text recognition.

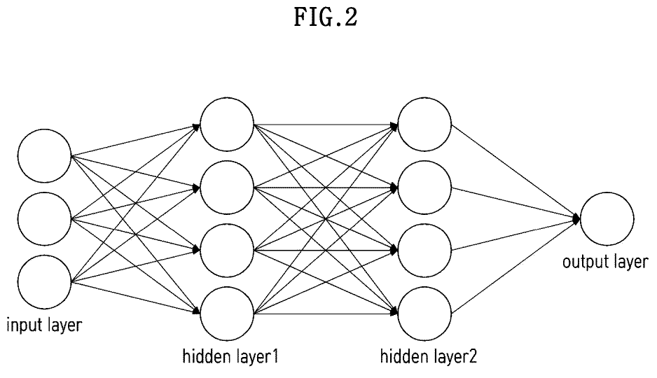

FIG. 2 is a view showing an example of a deep neural network.

A deep neural network (DNN) is an artificial neural network (ANN) having several hidden layers between an input layer and an output layer. The deep neural network is a machine learning model or an algorithm set referring to a deep neural network (DNN) having at least one hidden layer between an input layer and an output layer. Connection of the neural network is achieved from the input layer to the hidden layer and from the hidden layer to the output layer.

The deep neural network may model complex non-linear relationships, like a general artificial neural network. For example, in a deep neural network structure for an object identification model, each object may be expressed as a layer construction of basic elements of an image. At this time, additional layers may rally characteristics of lower layers that are gradually gathered. This characteristic of the deep neural network enables complex data to be modeled using fewer units (nodes) than an artificial neural network that is operated similarly thereto.

Previous deep neural networks were generally designed as feedforward neural networks. In recent research, however, deep learning structures have been successfully applied to a recurrent neural network (RNN). As an example, there are cases in which the deep neural network structure was applied to the field of language modeling. A convolutional neural network (CNN) has been well applied to the field of computer vision, and successful application cases have been well documented. Furthermore, in recent years, the convolutional neural network has been applied to the field of acoustic modeling for automatic recognition (ASR), and it is evaluated that the convolutional neural network has been more successfully applied than existing models. The deep neural network may be trained using a standard error back-propagation algorithm. At this time, weights may be updated through stochastic gradient descent.

Various signals from the surrounding environment received by a human through sense organs may be expressed through a computer in the form of text, audio, image, and video and stored as data in a storage device inside the computer.

High-dimensional data corresponding to the text, audio, image, and video stored in the computer is data including a combination of consecutive 0s and 1s from a low-dimensional perspective, and is various structures, objects, or class instances defined in a programming language used by each program from a slightly higher-dimensional perspective of a computer program.

For training by artificial intelligence technology so far, it is necessary to extract, from high-dimensional data such as text, audio, image, and video acceptable by humans through computers, features which are data that may effectively express the high-dimensional data, and an implementation method and terminology for such feature data are different among various artificial intelligence models and various programming languages that may implement the artificial intelligence models.

The present invention enables effective capture of inter-hemispheric interactions and spatiotemporal correlations of brain activity by forming a pair of EEG signals acquired from positions that are symmetric with respect to the midline of the skull, and by inputting the paired signals into a bidimensional empirical mode decomposition (BEMD) process. In particular, by converting the intrinsic mode functions (IMFs) extracted through BEMD into images including sampling-interval, time, amplitude, or frequency information, the nonlinear and nonstationary characteristics of the EEG signal can be represented more richly, thereby overcoming the limitations of conventional simple feature-extraction methods.

In addition, the present invention introduces a contrastive predictive coding (CPC)-based artificial intelligence model, which enables effective learning even in situations where labeled data are insufficient. Because CPC is trained to maximize mutual information between current and future inputs, it naturally captures the temporal continuity and contextual characteristics of emotional states and responds more flexibly to individual differences and context dependency. This effectively overcomes the data-dependence and generalization limitations of conventional supervised-learning approaches.

The following describes major terms used in the present invention.

### Empirical Mode Decomposition (EMD) and Intrinsic Mode Function (IMF)

Empirical mode decomposition is an adaptive data analysis method developed for analyzing nonlinear and nonstationary time-series data. Unlike Fourier or wavelet transforms, EMD has the advantage of decomposing an original signal into multiple intrinsic mode functions while preserving the local characteristics of the signal. Because EMD performs decomposition based on the inherent characteristics of the data itself, it is highly effective for analyzing complex signals exhibiting nonlinearity and nonstationarity.

An intrinsic mode function is a fundamental component obtained through the empirical mode decomposition process. An IMF must satisfy two conditions: (i) the numbers of extrema and zero-crossings across the entire dataset must be equal or differ by at most one, and (ii) the local mean value must be zero at any point in the signal. These conditions ensure that an IMF possesses a meaningful instantaneous frequency.

The core of EMD is the sifting process. All local maxima and minima of the original signal are first identified, and spline interpolation is applied to construct upper and lower envelopes. The mean of the two envelopes is subtracted from the original signal repeatedly until the resulting component satisfies the IMF conditions. This extracted IMF is removed from the original signal, and the same procedure is applied to the residual signal to extract subsequent IMFs.

The EMD method is widely used in various fields, including seismic-wave analysis, speech-signal processing, biomedical-signal analysis, and financial time-series analysis. It is particularly effective for extracting features of nonstationary signals and for noise reduction.

### Bidimensional Empirical Mode Decomposition (BEMD)

BEMD is an extension of the one-dimensional EMD method into two dimensions. It is designed for analyzing two-dimensional signals such as images or terrain data and decomposes an original two-dimensional signal into multiple intrinsic mode functions (IMFs) and a residual component. A primary characteristic of BEMD is that it identifies two-dimensional extrema and performs two-dimensional interpolation during decomposition, thereby allowing the decomposition process to take directional characteristics of the signal into account.

The sifting process of BEMD requires more complex computation than that of one-dimensional EMD. Local maxima and minima are identified on a two-dimensional plane, and two-dimensional spline interpolation is performed based on these points to form upper and lower envelope surfaces. The mean of the two envelope surfaces is subtracted from the original signal repeatedly until the resulting component satisfies the conditions of a two-dimensional IMF. BEMD can be applied in a variety of domains, including image compression, texture analysis, medical-image processing, and terrain analysis.

### Contrastive Predictive Coding (CPC)

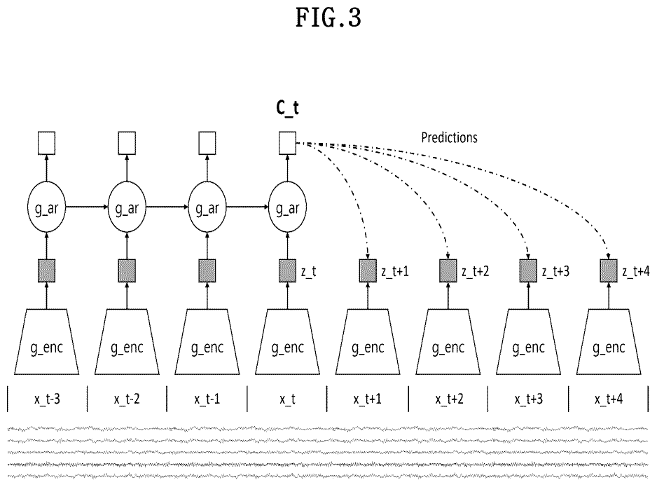

FIG. 3 is a diagram illustrating an example of a data processing procedure of a contrastive predictive coding (CPC)-based artificial intelligence model.

Referring to FIG. 3, the lower portion depicts EEG signals, which are directly processed by the CPC-based artificial intelligence model to classify an emotion of the user.

The basic structure of the CPC-based artificial intelligence model includes an encoder (g_enc), an autoregressive (AR) model (g_ar), and a prediction module. As shown in the lower part of FIG. 3, EEG signals are received as input and segmented at predetermined intervals for processing. The encoder converts each input sequence x_t into a latent representation (latent vector) z_t, effectively compressing and representing the complex spatiotemporal characteristics of the EEG signals. In particular, the encoder extracts important features of the input data, removes unnecessary noise, and preserves information beneficial for emotion classification.

The autoregressive model sequentially processes past latent representations to generate a context vector c t. Through this process, the model learns temporal changes and behavioral patterns associated with emotional states and focuses on understanding the context of the current time point by integrating information from previous time points. By modeling temporal dependencies among latent representations, the autoregressive model effectively captures the continuity and dynamic variation of emotional states.

The prediction module uses the current context vector c t to predict future latent representations z_t+k. Based on the current information, the model predicts multiple future time steps simultaneously, and this multi-step prediction helps the model learn richer temporal structures. To increase prediction accuracy, the model is trained to maximize mutual information between the current context and the actual future latent representations.

The training process of the CPC-based artificial intelligence model is performed in a self-supervised manner, allowing the model to learn temporal structure from data without requiring separate labeled information. To accurately predict future states based on the current state, the model automatically learns the essential characteristics and inherent patterns of the input data. The feature representations acquired through this process can then be effectively utilized in downstream tasks such as emotion classification. One significant advantage of the CPC-based model is its ability to achieve stable performance even in real-world environments where labeled data are scarce.

The present invention proposes an apparatus and method for classifying a user's emotion with high accuracy by using EEG signals processed through intrinsic mode functions (IMFs) and contrastive predictive coding (CPC).

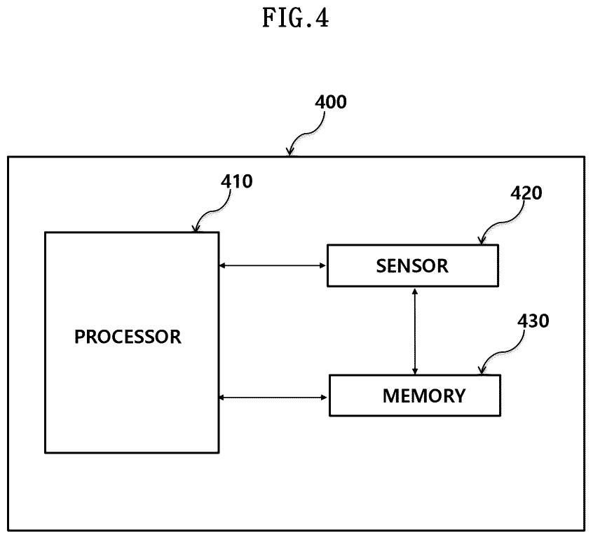

FIG. 4 is a block diagram illustrating the functional configuration of an EEG-based emotion classification apparatus 400 according to an embodiment of the present invention.

Referring to FIG. 4, the EEG-based emotion classification apparatus 400 may include a processor 410, a sensor 420, and a memory 430. The processor 410 is operably and/or electrically coupled to the sensor 420 and the memory 430 through one or more wired or wireless communication interfaces.

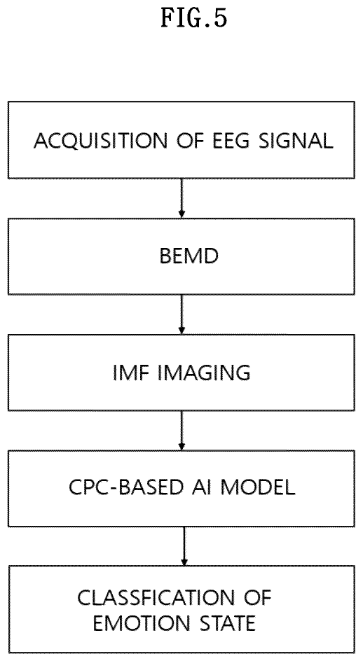

FIG. 5 is a diagram illustrating an example of a data processing procedure of the EEG-based emotion classification apparatus 400 according to an embodiment of the present invention.

The sensor 420 may acquire one or more electroencephalogram (EEG) signals of a user through one or more sensors, corresponding to the “EEG signal acquisition” stage in FIG. 5.

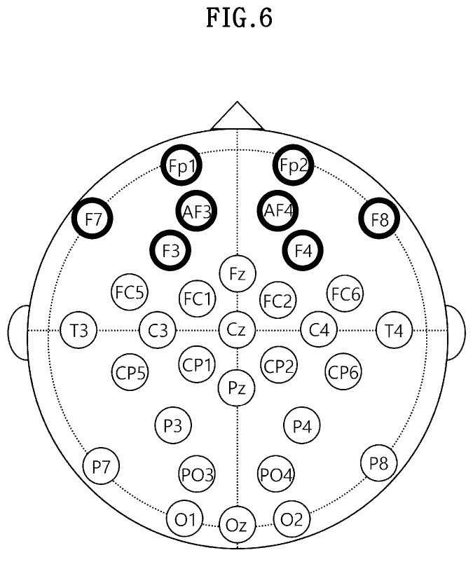

FIG. 6 is a diagram illustrating the structure of an International 10-20 electrode placement system.

Referring to FIG. 6, the International 10-20 electrode placement system is a standardized method for consistently attaching electrodes to the scalp for EEG measurement. This system enables noninvasive measurement of electrical activity of the brain, and electrode positions are determined by placing electrodes at intervals corresponding to 10% or 20% of the distances between anatomical landmarks of the skull, such as the nasion, the inion, and the preauricular points.

The naming of each electrode is expressed as a combination of an alphabet and a numerical value that represents the corresponding region of the brain. ‘F denotes the frontal lobe, ‘T’ the temporal lobe, ‘C’ the central region, the parietal lobe, and ‘O’ the occipital lobe. Odd numbers refer to the left hemisphere, even numbers to the right hemisphere, and ‘z’ indicates the midline. Moreover, ‘Fp’ indicates the prefrontal area, and ‘AF’ indicates the anterior frontal area. This systematic naming convention allows for intuitive identification of electrode positions.

One major advantage of this system lies in the reproducibility and standardization of electrode placement. Because electrodes can be positioned according to the same proportional scheme regardless of differences in the size or shape of the skull among subjects, the comparison and interpretation of test results become significantly easier. The uniform spacing of electrodes enables comprehensive monitoring of electrical activity across the brain, which is particularly important in diagnosing and studying neurological disorders such as epilepsy and sleep disorders. This standardized system is widely used in clinical and research settings worldwide.

The sensor 420 may acquire EEG signals of a user from at least one region among the regions corresponding to Fp1, Fp2, AF3, AF4, F3, F4, F7, and F8, which are indicated with a bold outline in FIG. 5. These regions-Fp1, Fp2, AF3, AF4, F3, F4, F7, and F8—are known areas for effectively measuring EEG activity associated with human emotions.

Preferably, the sensor 420 may acquire EEG signals of a user from the total of eight regions corresponding to Fp1, Fp2, AF3, AF4, F3, F4, F7, and F8, or from a region including the above eight regions, as indicated with a bold outline in FIG. 6.

The processor 410 may perform a bidimensional empirical mode decomposition (BEMD) process on the acquired one or more EEG signals to extract one or more intrinsic mode functions (IMFs), corresponding to the “BEMD” processing stage in FIG. 5.

TO effectively capture inter-hemispheric interactions and spatiotemporal correlations of the brain, the processor 410 may form pairs of EEG signals acquired from positions that are symmetric with respect to the midline of the skull among the EEG signals obtained through the sensor 420, and may perform the BEMD process on the paired EEG signals.

For example, when the sensor 420 may acquire the user's EEG signals from the total of eight regions corresponding to Fp1, Fp2, AF3, AF4, F3, F4, F7, and F8, as indicated with a bold outline in FIG. 6, the processor 410 may form pairs such as Fp1-Fp2, AF3-AF4, F3-F4, and F7-F8, input the paired signals to the BEMD process, and extract corresponding IMFs for each pair.

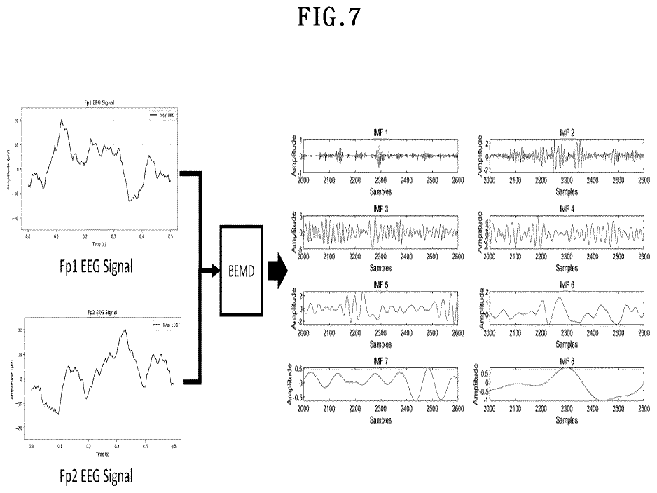

FIG. 7 is a diagram illustrating an example of a bidimensional empirical mode decomposition (BEMD) process of the EEG-based emotion classification apparatus 400 according to the present invention.

Referring to FIG. 7, the processor 410 forms a pair of EEG signals acquired from the Fp1 and Fp2 regions of the International 10-20 electrode placement system and extracts a total of eight IMFs through the BEMD process. The number of IMFs may vary depending on the waveform characteristics of the paired EEG signals input to the BEMD process.

The processor 410 may convert signal data of each of the extracted one or more IMFs into one or more images based on the IMFs, corresponding to the “IMF imaging” stage of FIG. 5. In this case, the processor 410 may convert signal data of each of the extracted IMFs into one or more images including at least one of sampling interval, time, amplitude, and frequency.

Referring to FIG. 7, the processor 410 may convert signal (signal data) of each of the eight IMFs extracted through the BEMD process into an image including amplitude (Y-axis) information with respect to sampling interval (X-axis) and may also convert signal (signal data) of each IMF into an image including frequency (Y-axis) information with respect to sampling time (X-axis).

In the present invention, the processor 410 does not directly use the IMFs extracted through BEMD but instead utilizes images converted from the IMFs. This is because the signal patterns inherent in IMFs have complex structural characteristics, making direct learning difficult. By using image-based processing, more precise emotion classification can be achieved, which enables effective analysis and interpretation of complex signal patterns.

The processor 410 may apply the converted one or more images to a predetermined trained contrastive predictive coding (CPC)-based artificial intelligence model to classify the user's emotion, corresponding to the “CPC-based artificial intelligence model” and “emotion-state classification” stages of FIG. 5.

For example, when the processor 410 forms pairs Fp1-Fp2, AF3-AF4, F3-F4, and F7-F8, inputs each pair to the BEMD process, and extracts eight IMFs from each pair, the processor 410 may extract a total of thirty-two IMFs and generate thirty-two IMF images, which may then be applied to the CPC-based artificial intelligence model.

The predetermined trained CPC-based artificial intelligence model may process the input IMF images and output probability values corresponding to predefined emotion categories. Based on the output probability values, the processor 410 may determine the emotional state of the user by selecting the emotion category having the highest probability.

Although not shown in FIG. 5, the processor 410 may perform at least one preprocessing operation such as noise removal, frequency filtering, or signal normalization-on the one or more EEG signals acquired through the sensor 420 prior to performing the BEMD process.

The memory 430 may store one or more EEG signals of the user acquired through the sensor 420, the BEMD algorithm, the predetermined trained CPC-based artificial intelligence model, one or more IMFs and IMF images, emotion-classification results, and various computation-related information generated through the processor 410.

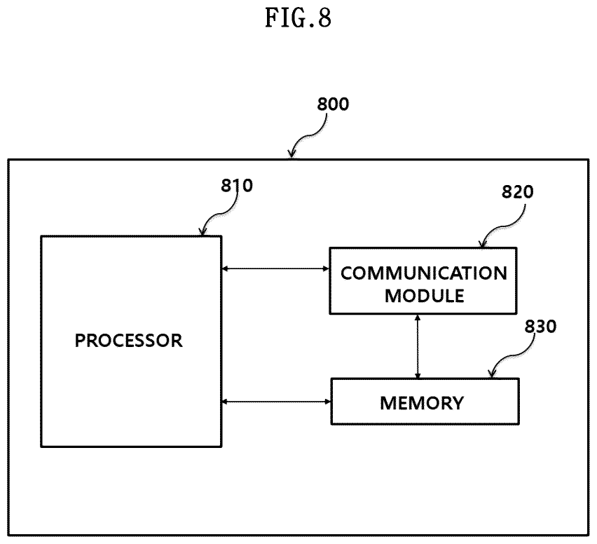

FIG. 8 is another exemplary diagram illustrating a block diagram of an EEG-based emotion classification apparatus 800 according to the present invention.

Referring to FIG. 8, the sensor 420 included in the EEG-based emotion classification apparatus 400 of FIG. 4 may be replaced with a communication module 820.

The communication module 820 may receive EEG signals of a user from at least one region among the regions corresponding to Fp1, Fp2, AF3, AF4, F3, F4, F7, and F8, which are indicated with a bold outline in FIG. 5, and this corresponds to an “EEG signal reception” operation replacing the “EEG signal acquisition” process in FIG. 5. The communication module 820 may receive EEG signals of a user from at least one of these regions, which are known to be associated with human emotional activity.

Preferably, the communication module 820 receives the user's EEG signals from the total of eight regions-Fp1, Fp2, AF3, AF4, F3, F4, F7, and F8—or from a region including these eight regions, as indicated with a bold outline in FIG. 6.

The processor 810 may perform a bidimensional empirical mode decomposition (BEMD) process on the one or more EEG signals received through the communication module 820 to extract one or more intrinsic mode functions (IMFs), corresponding to the “BEMD” stage of FIG. 5.

To effectively capture inter-hemispheric interactions and spatiotemporal correlations of the brain, the processor 810 may form pairs of EEG signals that are acquired from positions symmetric with respect to the midline of the skull and may perform the BEMD process on the paired EEG signals.

For example, when the communication module 820 receives the user's EEG signals obtained from the total of eight regions-Fp1, Fp2, AF3, AF4, F3, F4, F7, and F8—as indicated in FIG. 6, the processor 810 may form the pairs Fp1-Fp2, AF3-AF4, F3-F4, and F7-F8, input the paired signals to the BEMD process, and extract corresponding IMFs for each pair.

Referring to FIG. 7, the processor 810 forms a pair of EEG signals acquired from the Fp1 and Fp2 regions of the International 10-20 system and extracts a total of eight IMFs through the BEMD process. The number of IMFs may vary depending on the waveform characteristics of the paired EEG signals input to the BEMD process.

The processor 810 may convert signal data of each of the extracted one or more IMFs into one or more images, corresponding to the “IMF imaging” stage of FIG. 5. In doing so, the processor 810 may convert signal data of each of the extracted IMFs into one or more images including at least one of sampling interval, time, amplitude, and frequency.

Referring again to FIG. 7, the processor 810 may convert signal data of each of the eight IMFs extracted through the BEMD process into an image including amplitude (Y-axis) with respect to sampling interval (X-axis), and may also convert each IMF into an image including frequency (Y-axis) with respect to sampling time (X-axis).

In the present invention, the processor 810 is configured not to use the IMFs extracted through BEMD directly but to utilize images converted from the IMFs. This is because IMFs inherently contain structurally complex signal patterns that make direct learning difficult. The image-based processing approach enables more precise emotion classification and provides an advantage in effectively analyzing and interpreting complex signal patterns.

The processor 810 may apply the converted one or more images to a predetermined trained contrastive predictive coding (CPC)-based artificial intelligence model to classify the user's emotional state, corresponding to the “CPC-based artificial intelligence model” and “emotion-state classification” stages of FIG. 5.

For example, when the processor 810 may form the pairs Fp1-Fp2, AF3-AF4, F3-F4, and F7-F8, inputs each pair to the BEMD process, and extracts eight IMFs per pair, the processor 810 may extract a total of thirty-two IMFs and generate thirty-two corresponding IMF images, which may then be provided to the CPC-based artificial intelligence model.

The predetermined trained CPC-based artificial intelligence model may process the IMF images provided as input and output probability values corresponding to predefined emotion categories. Based on the output probability values, the processor 810 may determine the user's emotion by selecting the emotion category having the highest probability.

Although not illustrated in FIG. 5, the processor 810 may perform at least one preprocessing operation-such as noise removal, frequency filtering, or signal normalization-on the one or more EEG signals received through the communication module 820 prior to performing the BEMD process.

The memory 830 may store one or more EEG signals of the user received through the communication module 820, the BEMD algorithm, the predetermined trained CPC-based artificial intelligence model, one or more IMFs and IMF images, emotion-classification results, and various computation-related information generated by the processor 810.

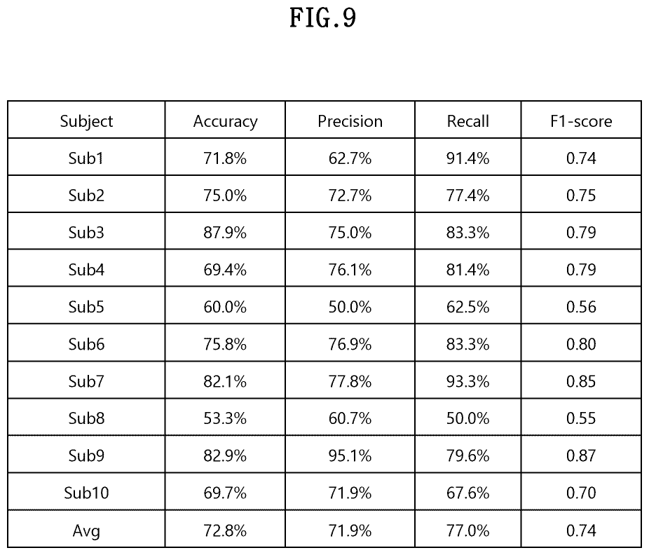

FIG. 9 is a diagram illustrating performance evaluation results of the EEG-based emotion classification apparatuses 400 and 800 according to the present invention.

The performance evaluation results shown in FIG. 9 compare intra-subject performance for each individual. The intra-subject evaluation method performs model training and evaluation independently for each subject using only that subject's data. This method is effective for measuring performance while reflecting EEG signal characteristics unique to each individual.

In the performance evaluation results illustrated in FIG. 9, 70% of each subject's EEG data was used for training the CPC-based artificial intelligence model, 20% was used as fine-tuning data, and the remaining 10% was used as test data. For quantitative evaluation of the model, the metrics Accuracy, Precision, Recall, and F1-score were used.

Accuracy is a metric that represents the proportion of correct predictions among all predictions. It is calculated as the sum of True Positives and True Negatives divided by the total number of data samples and is the most intuitive and commonly understood evaluation indicator.

Precision is a metric representing the proportion of samples predicted as positive that are actually positive. It is calculated by dividing True Positives by the sum of True Positives and False Positives. Precision is especially important in applications where False Positives carry significant risk, such as spam filtering or fraudulent transaction detection.

Recall is a metric representing the proportion of actual positive samples that are correctly predicted as positive by the model. It is calculated by dividing True Positives by the sum of True Positives and False Negatives. Recall is particularly important in fields where False Negatives must be minimized, such as disease diagnosis or defect detection.

The F1-score is a comprehensive evaluation metric calculated as the harmonic mean of Precision and Recall. It is computed by multiplying Precision and Recall by two and dividing by their sum. The F1-score provides a balanced performance assessment even in the presence of data imbalance and is one of the most widely used performance indicators in real-world applications.

As shown in FIG. 9, the performance evaluation results of the EEG-based emotion classification apparatuses 400 and 800 according to the present invention demonstrate high performance across all metrics-Accuracy, Precision, Recall, and F1-score.

The present invention provides an apparatus and method for classifying a user's emotional state with high accuracy by using intrinsic mode functions (IMFs) and contrastive predictive coding (CPC) applied to EEG signals.

By processing EEG signals acquired from positions that are symmetric with respect to the midline of the skull through BEMD, the present invention enables effective capture of inter-hemispheric interactions and spatiotemporal correlations of brain activity. In particular, by converting signal data of each of IMFs extracted through BEMD into one or more images that include sampling-interval, time, amplitude, and frequency information, the nonlinear and nonstationary characteristics of EEG signals can be expressed in a richer manner, thereby successfully overcoming the limitations of conventional simple feature-extraction approaches.

The present invention significantly improves the accuracy and reliability of emotion classification by combining a CPC-based artificial intelligence model with an advanced signal-processing technique based on BEMD. The invention is capable of flexibly responding to individual differences in emotional expression and context-dependent variations in signal patterns, and it also enables effective learning in environments where labeled data are insufficient, thereby greatly enhancing applicability in real-world settings.

The effects obtainable from the present invention are not limited to those described above. Other effects not explicitly mentioned will be apparent to those skilled in the art from the following description.

The apparatus described above may be implemented using hardware components, software components, and/or a combination of hardware and software components. For example, the apparatus and components described in the embodiments may be implemented using one or more general-purpose or special-purpose computers capable of executing and responding to instructions, such as a processor, controller, ALU (arithmetic logic unit), digital signal processor, microcomputer, FPGA (field-programmable gate array), PLU (programmable logic unit), microprocessor, or any other device capable of executing instructions. The processing device may execute an operating system (OS) and one or more software applications running on the operating system. In response to software execution, the processing device may access, store, manipulate, process, and generate data. For ease of understanding, certain descriptions herein refer to the processing device in the singular; however, those skilled in the art will appreciate that the processing device may include multiple processing elements and/or multiple types of processing elements. For example, the processing device may include multiple processors or a combination of a processor and a controller. Other processing configurations, such as a parallel processor, are also possible.

Software may include a computer program, code, instructions, or any combination thereof, and may configure or collectively instruct the processing device to operate in a desired manner. Software and/or data may be embodied permanently or temporarily on any type of machine, component, physical device, virtual apparatus, computer-readable storage medium, or signal wave, to allow interpretation by the processing device or to provide instructions or data thereto. Software may also be distributed across network-connected computing devices and stored or executed in a distributed manner. Software and data may be stored on non-transitory computer-readable storage medium or one or more computer-readable recording media.

Methods according to embodiments may be implemented as program instructions executable through various computer means and may be recorded on a computer-readable medium. The computer-readable medium may include program instructions, data files, data structures, or a combination thereof. The program instructions recorded on the medium may include those specially designed and configured for the embodiments or those known and available to those skilled in the art of computer software. Examples of computer-readable recording media include magnetic media such as hard disks, floppy disks, and magnetic tapes, optical media such as CD-ROMs and DVDs, magneto-optical media such as floptical disks; and hardware apparatuses specially configured to store and execute program instructions, such as ROM, RAM, and flash memory. Program instructions include machine-language code generated by a compiler as well as higher-level language code executable by a computer using an interpreter or the like. The hardware devices may be configured to operate as one or more software modules to perform the operations of the embodiments, and vice versa.

The embodiments described above represent combinations of components and features in predetermined forms. Each component or feature should be considered optional unless expressly stated otherwise. Each component or feature may be implemented without being combined with other components or features. Additionally, combinations of some components and/or features may constitute another embodiment of the invention. The order of operations described in the embodiments may be changed. Some components or features of one embodiment may be included in another embodiment for replaced with corresponding components or features of another embodiment. It is apparent that claims not explicitly recited as dependent in the claim set may nonetheless be combined to form various embodiments or may be incorporated as new claims through amendments after filing. The processors 410, 810 according to the present

invention may be implemented by hardware, firmware, software, or a combination thereof. When implemented using hardware, the processors 410, 810 may include ASICS (application-specific integrated circuits), DSPs (digital signal processors), DSPDs (digital signal processing devices), PLDs (programmable logic devices), FPGAS (field-programmable gate arrays), or other circuits configured to perform the operations of the invention. The present invention may also be embodied as a computer-readable recording medium storing a program for executing the method for preventing leakage of user information during user authentication.

The present invention may be embodied in other specific forms without departing from the essential characteristics of the invention, as will be apparent to those skilled in the art. Therefore, the foregoing detailed description is to be considered exemplary rather than limiting in all respects. The scope of the invention should be determined by the reasonable interpretation of the appended claims, and all modifications within the equivalent scope of the invention are to be included therein.

What is claimed is:

1. An emotion classification apparatus, comprising:
a sensor configured to acquire one or more electroencephalogram (EEG) signals of a user; and
a processor operably coupled to the sensor, and configured to:
perform a bidimensional empirical mode decomposition (BEMD) process on the acquired one or more EEG signals to extract one or more intrinsic mode functions (IMFs),
convert signal data of each of the extracted IMFs into one or more images, and
classify an emotion of the user by applying the converted one or more images to a certain trained contrastive predictive coding (CPC)-based artificial intelligence model.

2. The emotion classification apparatus of claim 1, wherein the sensor is further configured to acquire the EEG signals from at least one region corresponding to Fp1, Fp2, AF3, AF4, F3, F4, F7, and F8 of an International 10-20 electrode placement system.

3. The emotion classification apparatus of claim 1, wherein the processor is further configured to perform the BEMD process by pairing EEG signals acquired from positions symmetrical with respect to a midline of a skull among the one or more EEG signals acquired through the sensor.

4. The emotion classification apparatus of claim 1,
wherein the processor is further configured to convert the extracted IMFs into an image including at least one of a sampling interval, time, amplitude, and frequency.

5. The emotion classification apparatus of claim 1,
wherein the processor is further configured to perform at least one preprocessing operation selected from a noise removal, a frequency filtering, and a signal normalization on the one or more EEG signals acquired through the sensor prior to performing the BEMD process.

6. An emotion classification apparatus, comprising:
a communication module configured to receive one or more electroencephalogram (EEG) signals of a user; and
a processor operably coupled to the communication module, and configured to:
perform a bidimensional empirical mode decomposition (BEMD) process on the received one or more EEG signals to extract one or more intrinsic mode functions (IMFs);
convert signal data of each of the extracted IMFs into one or more images; and
classify an emotion of the user by applying the converted one or more images to a certain trained contrastive predictive coding (CPC)-based artificial intelligence model.

7. A method for emotion classification by an emotion classification apparatus, comprising:
acquiring one or more electroencephalogram (EEG) signals of a user;
performing a bidimensional empirical mode decomposition (BEMD) process on the acquired one or more EEG signals to extract one or more intrinsic mode functions (IMFs);
converting signal data of each of the extracted IMFs into one or more images; and
classifying an emotion of the user by applying the converted one or more images to a certain trained contrastive predictive coding (CPC)-based artificial intelligence model.

8. The method of claim 7, wherein the acquiring comprises acquiring the EEG signals from at least one region corresponding to Fp1, Fp2, AF3, AF4, F3, F4, F7, and F8 of an International 10-20 electrode placement system.

9. The method of claim 7, wherein the performing comprises performing the BEMD process by pairing EEG signals acquired from positions symmetrical with respect to a midline of a skull among the one or more EEG signals.

10. The method of claim 7, wherein the converting comprises converting the extracted IMFs into an image including at least one of a sampling interval, time, amplitude, and frequency.

11. The method of claim 7, further comprising:
performing at least one preprocessing operation selected from a noise removal, a frequency filtering, and a signal normalization on the one or more EEG signals acquired through the sensor prior to performing the BEMD process.

12. A method for emotion classification by an emotion classification apparatus, comprising:
receiving one or more electroencephalogram (EEG) signals of a user;
performing a bidimensional empirical mode decomposition (BEMD) process on the received one or more EEG signals to extract one or more intrinsic mode functions (IMFs);
converting signal data of each of the extracted IMFs into one or more images; and
classifying an emotion of the user by applying the converted one or more images to a certain trained contrastive predictive coding (CPC)-based artificial intelligence model.

13. A non-transitory computer-readable storage medium storing instructions that, when executed by one or more processors, cause the one or more processors to:
acquire one or more electroencephalogram (EEG) signals of a user;
perform a bidimensional empirical mode decomposition (BEMD) process on the acquired one or more EEG signals to extract one or more intrinsic mode functions (IMFs);
convert signal data of each of the extracted IMFs into one or more images; and
classify an emotion of the user by applying the converted one or more images to a certain trained contrastive predictive coding (CPC)-based artificial intelligence model.

14. A non-transitory computer-readable storage medium storing instructions that, when executed by one or more processors, cause the one or more processors to:
receive one or more electroencephalogram (EEG) signals of a user;
perform a bidimensional empirical mode decomposition (BEMD) process on the received one or more EEG signals to extract one or more intrinsic mode functions (IMFs);
convert signal data of each of the extracted IMFs into one or more images; and
classify an emotion of the user by applying the converted one or more images to a certain trained contrastive predictive coding (CPC)-based artificial intelligence model.
